(19) United States
(12) Patent Application Publication
(10) Pub. No.: US 2016/0002588 A1
(43) Pub. Date: Jan. 7, 2016

(54) SOLID PHASE BASED HIGH YIELD BIOFUEL TECHNOLOGY

(71) Applicant: Immunotrex Biologics, Inc., North Andover, MA (US)

(72) Inventor: Pei Zhang, Malden, MA (US)

(21) Appl. No.: 14/738,419

(22) Filed: Jun. 12, 2015

Related U.S. Application Data
(60) Provisional application No. 62/011,378, filed on Jun. 12, 2014.

(51) Int. Cl. C12N 1/12

(52) U.S. Cl. CPC C12N 1/12

(57) ABSTRACT

A method of solid phase based technology is provided for enhanced growth of microorganisms such as cyanobacteria and algae for biofuel production. An improved growth rate and cell density of cyanobacteria is exhibited.

### CROSS REFERENCE TO RELATED APPLICATION

This application claims the benefit of provisional application Ser. No. 62/011,378, filed Jun. 12, 2014, which is incorporated herein by reference in its entirety.

### BACKGROUND OF THE INVENTION

Cyanobacteria and algae use solar energy to convert carbon dioxide into carbohydrates, lipids and other molecules that can be processed into biofuels. Cyanobacteria exhibit higher solar conversion efficiency and growth rates than plants and eukaryotic microalgae. There is increased interest in engineering cyanobacteria to convert carbon dioxide into a desirable fuel. However, large scale cultivation of cyanobacteria requires a large physical area, substantial water usage and the resultant energy yield is low. It is desirable to develop efficient, high yield systems for the production of cyanobacteria biomass.

### SUMMARY OF THE INVENTION

A unique method of solid phase based culturing technology generating higher yield production of cyanobacteria and algae is provided. Enhanced cell density and correlated growth rate of cyanobacteria is exhibited.

A method for enhancing the growth and replication of microorganisms, in some embodiments, cyanobacteria or specific species of algae is provided. Cyanobacteria and algae can serve as novel sources for the production of large quantities of long-chain insoluble hydrocarbons (e.g., alkenones) useful for the production of commercial hydrocarbons such as methane fuels and chemicals. In embodiments, a method for increasing the growth rate and/or yield of cyanobacteria or specific species of algae in an aqueous culture medium is provided comprising introducing a solid phase material into the aqueous culture medium. In some embodiments, the membrane is introduced after inoculating the culture medium with the cyanobacteria. In some embodiments, the membrane is introduced prior to inoculating the culture medium with the cyanobacteria. In some embodiments, the solid phase material in the medium is inoculated with the cyanobacteria.

In some embodiments, the cyanobacteria is selected from one or more of the group consisting of Synechocystis sp. PCC 6803, Anabaena variabilis ATCC 29413, Anabaena variabilis PK84, Anabaena variabilis PK17, Crocosphaera watsonii WH 8501, Cyanothece sp. ATCC 51142, Cyanothece sp. PCC 7424, Cyanothece sp. PCC 7425, Cyanothece sp. PCC 8801, Cyanothece sp. PCC 8802, Synechococcus sp. JA-3-3Ab, Synechococcus sp. JA-2-3B′a(2-13), Microcystis aeruginosa NIES-843, Synechococcus sp. CC9311, Synechococcus sp. CC9605, Synechococcus sp. CC9902, Synechococcus sp. PCC 7002, Synechococcus sp. RCC307, Synechococcus sp. WH 7803, Synechococcus elongatus PCC 6301, Synechococcus elongatus PCC 7942, Synechococcus sp. WH 8102, Thermosynechococcus elongatus BP-1, Spirulina spp. and cyanobacterium UCYN-A. In some embodiments, the cyanobacteria is selected from Synechocystis sp. PCC 6803, and Anabaena variabilis PK84. Algae sp.

In some aspects, the cyanobacteria is selected from one or more of the group consisting of Synechocystis sp. PCC 6803, Anabaena variabilis ATCC 29413, Anabaena variabilis PK84, and Anabaena variabilis PK17.

In some embodiments, cyanobacteria or algae is co-cultured with other species in syntrophic growth. In some other embodiment, mixed culture of Synechocystis sp. or other pure culture photosynthetic microorganisms with E. coli sp. or other oxygen respiring heterotrophic or autotrophic pure culture microorganisms are co-cultured in the membrane.

In some embodiments the solid phase material is selected from a porous non-woven, woven, knitted, or foam material.

In some embodiments, the non-woven material is selected from e.g., polyethylene, polyethylene blend, polyester, polyester blend, rayon, rayon blend, fiberglass, polypropylene (PP), polypropylene blend, fiberglass-polypropylene blend, fiberglass blend, polyethylene (PET), polyethylene blend; fiberglass-polyethyene blend; polypropylene-fiberglass blend; rayon-polyethylene blend; wool; wool blend; nylon; nylon blend.

In some embodiments, the woven or knitted material is elected from nylon, polyester, polypropylene or PEEK porous material.

In some embodiments, the solid phase material is an open-cell foam material selected from polyurethane, or reticulated polyurethane foam.

In some embodiments, the solid phase is purged or flushed by CO₂ to provide high yield.

In some embodiments, the geometric design of the solid phase material is in cylinder or block shape with a hollow in center, for example, as shown in FIG. 3B.

In some embodiments, a hydrophobic gas exchange membrane is employed that separates the reactor from the atmosphere in order to provide uniform gas exchange for high yield with moderate water evaporation.

In some embodiments, the solid phase material is treated with Teflon or Nafion or other pure or mixed reagents (soaking and baking) to have changed the surface hydrophobicity.

In some embodiments, the solid phase culture is produced in a medium is selected from Aiba and Ogawa (AO) Medium, Allen Medium, Allen and Amon Medium plus Nitrate: ATCC Medium 1142, Antia's (ANT) Medium, Aquil Medium, Ashbey's Nitrogen-free Agar, ASN-III Medium, ASNIII+Turks Island Salts: CRBIP Medium 1538, ASP 2 Medium, ASW Medium: Artificial Seawater and Derivatives, ATCC Medium 617: BG-11 for Marine Blue-Green Algae; Modified ATCC Medium 616 [BG-11 medium], ATCC Medium 819: Blue-green Nitrogenfixing Medium; ATCC Medium 616 [BG-11 medium] without NO3, ATCC Medium 854: ATCC Medium 616 [BG-11 medium] with Vitamin B12, ATCC Medium 1047: ATCC Medium 957 [MN marine medium] with Vitamin B12, ATCC Medium 1077: Nitrogen-fixing marine medium; ATCC Medium 957 [MN marine medium] without NO3, ATCC Medium 1234: BG-11 Uracil medium; ATCC Medium 616 [BG-11 medium] with Uracil, Beggiatoa Medium: ATCC Medium 138, Beggiatoa Medium 2: ATCC Medium 1193, Blue-Green (BG) Medium, BG-11 Medium for Blue Green Algae: ATCC Medium 616, BG11+ASNIII (10%): CRBIP Medium 1540, BG11+ASNIII (1:1): CRBIP Medium 1546, BG11+NaHCO3: CRBIP Medium 1547; BG11+Turks Island Salts (25%)+NaHCO3:CRBIP Medium 1548, Bold's Basal (BB) Medium, Bold 1NV Medium, Bold 3N Medium, Bristol Medium, Castenholtz D Medium, Castenholtz D Medium Modified: Halophilic Cyanobacteria, Castenholtz DG Medium, Castenholtz DGN Medium, Castenholtz ND Medium, Chloroflexus Broth, Chloroflexus Medium: ATCC Medium 920, Chu's #10 Medium: ATCC Medium 341, Chu's #10 Medium Modified, Chu's #11 Medium Modified, COMBO Medium Modified, CR1 Soil, Cyanophacyean Medium, DCM Medium, DYIV Medium, E27 Medium, E31 Medium and Derivatives, Erd-Schreiber 2× Medium, f/2 Medium, f/2 Medium Derivatives, Fraquil Medium: Freshwater Trace Metal-Buffered Medium, Gorham's Medium for Algae: ATCC Medium 625, h/2 Medium, Jansen's (J) Medium, Jaworski's (JM) Medium, K Medium, L1 Medium and Derivatives, MN Marine Medium: ATCC Medium 957, Plymouth Erdschreiber (PE) Medium, Prochlorococcus PC Medium, Prochlorococcus Medium: CRBIP Medium 1559, Pro99 Medium, Proteose Peptone (PP) Medium, Prov Medium, Prov Medium Derivatives, S77 plus Vitamins Medium, S88 plus Vitamins Medium, Saltwater Nutrient Agar (SNA) Medium and Derivatives, SES Medium, SN Medium, Modified SN Medium, SNAX Medium, Soil/Water Biphasic (S/W) Medium and Derivatives, SOT Medium for Spirulina: ATCC Medium 1679, Spirulina (SP) Medium, van Rijn and Cohen (RC) Medium, Walsby's Medium, YBC-II Medium, Yopp Medium, and Z8 Medium.

In some embodiments, the medium is BG-11 medium ATCC 616.

In some embodiments, a method for increasing growth rate and/or yield of cyanobacteria in an aqueous culture is provided, the method comprising introducing a solid phase material into the aqueous culture in a photobioreactor.

In some embodiments, the cell culture is directly inoculated into solid phase by injecting cell culture into the pores of the solid phase material.

In some embodiments, the cell culture is inculcated into liquid phase surrounding the solid phase material. The cells will migrate and colonized in solid phase through natural augmentation.

In some aspects, the photobioreactor is fitted with a means for providing irradiated light, for example, between about 400 nm to 770 nm wavelength. In other aspects, the photoreactor is fitted with a means for introducing carbon dioxide to the aqueous culture medium.

In some aspects, the photobioreactor utilizes sunlight without extra light sources.

In some embodiment, CO₂ is purged into center of the solid phase in the photobioreactor and diffused outwards from the center to the substrate solution surrounding the solid phase (FIG. 3B).

### BRIEF DESCRIPTION OF THE DRAWINGS

FIG. 1 Comparison of Time-lapse cell density of Solid Phase based High Yield method (SPHY) to control. The control refers to the cultures that are without solid phase. The results of SPHY combine the cell density in solid phase and in liquid phase of the culture. SPHY dimension is 20 mm×20 mm×5 mm. At day 18, solid phase material was taken out of the culture vessel and treated by purging with CO₂ for 1 hour while the control stays the same condition. All samples are propagated under the same conditions. Blank was deducted from all data. Error bars were calculated at the 95% confidence interval. At day 21, 3 days after CO₂ purging, resultant SPHY combined solid and liquid phase cell density have a significant increase compared to control culture grown under the same conditions without the solid phase material.

FIG. 2 Comparison of Time-lapse cell density of Solid Phase based High Yield method (SPHY) to control. The control refers to the cultures that are without solid phase. The results of cell density of SPHY in solid phase were separated from liquid phase. SPHY dimension is 20 mm×20 mm×5 mm. At day 18, solid phase material was taken out of the culture vessel and treated by purging with CO₂ for 1 hour while the control stays the same condition. All samples are propagated under the same conditions. Blank was deducted from all data. Error bars were calculated at the 95% confidence interval. SPHY solid phase culture and resulted combined solid and liquid phase cell density have a significant increase compared to control culture grown under the same conditions without the solid phase material. At day 21, 3 days after CO₂ purging, the separated SPHY solid phase culture and liquid phase culture both have a significant increase of cell density compared to control culture grown under the same conditions without the solid phase material.

FIG. 3A shows a schematic of one example bioreactor for solid phase based production of cyanobacteria.

FIG. 3B shows one geometric design of the solid phase material with a hollow center.

FIG. 4A shows pure Cyanobacteria Synechocystis PCC 6803 culture prepared according to Example 1 in a 50 mL conical centrifuge tube. Cells grow in higher yield using the solid phase based high yield technique according to example 1 on the left; compared to control culture under identical conditions, but without the solid phase material, on the right. Both tubes were treated under the same conditions for three days, measuring optical density periodically. After 3 days, in the solid phase portion of the culture, cells have 40% higher yield than the control. After 3 days, in the liquid phase portion of the culture, cells have 14% higher yield than the control.

FIG. 4B shows a culture grown in a 250 mL Erlenmeyer flask with solid phase material freely floating in the culture. The flask is shown partially submerged in an incubator water bath with exposure to light from above.

### DETAILED DESCRIPTION OF THE INVENTION

In describing and claiming the present invention, the following terminology will be used in accordance with the definitions set forth below.

The singular forms “a,” “an,” and “the” include plural referents unless the context clearly dictates otherwise. Thus, for example, reference to “an alga” includes reference to one or more of such algae, and reference to “the cover” includes reference to one or more of such covers.

As used herein, the term “biomass” refers to various carbon-containing materials resulting from growth of cyanobacteria or algae, but may include material from other growing organisms. In some aspects, a “biomass” can consist of various carbon containing materials resulting from the growth cyanobacteria, algae, and other microbial species. In some aspects, the term “biomass” can also include reference to only the living organisms themselves when indicated by the context used herein.

As used herein, the term “substantially” refers to the complete or nearly complete extent or degree of an action, characteristic, property, state, structure, item, or result. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be +/−10% of the endpoint.

Efficient methods for growing cyanobacteria in an aqueous media comprising a solid phase material are provided. Without being bound by theory, the membrane offers surface properties conducive to growth of the cyanobacteria. The media may offer a place for the cyanobacteria to attach. The hydrophobic properties of the membrane and/or the pore sizes of the membrane may serve to enhance the growth curve, and allow higher growth rate and overall yields of the cyanobacteria.

Cyanobacteria or specific species of algae use solar energy to convert carbon dioxide into large quantities of long-chain insoluble hydrocarbons (alkenones) for the production of commercial hydrocarbons such as methane fuels and chemicals

Cyanobacteria exhibit higher solar conversion efficiency and growth rates than plants and eukaryotic microalgae.

Cyanobacteria and/or algae are grown to produce biomass for biofuel production, or are genetically manipulated to produce secretable products directly, for example, ethanol, ethylene, isoprene, free fatty acids, fatty alcohols, isobutyraldehyde, 1-butanol, or hydrogen.

Algae, for example, can be converted into various types of fuel. The lipid, or oily part of the algae biomass can be extracted and converted into biodiesel through a process similar to that used for any other vegetable oil, or converted in a refinery into replacements for petroleum-based fuels. Alternatively, or following lipid extraction, the carbohydrate content of algae can be fermented into biofuel, for example, ethanol or butanol.

One advantage of the genetically manipulated cyanobacteria or algae, is that several costly process steps for production of biodiesel and green diesel, such as solvent extraction.

One of the most efficient biomass production methods involves the growth of cyanobacteria. For example, one method of producing usable fuels from biomass is to gasify biomass. Gasification can be accomplished by various methods, including pyrolysis, catalytic hydrothermal gasification, and anaerobic digestion.

Regardless of the microorganism species, the ability to produce the biomass feedstock as cheaply and quickly as possible is important to achieve the most efficient production of biofuel. In addition, it is desirable to achieve the highest level of production with minimal use of resources such as nutrients, water, or light.

Methods disclosed herein for efficient production of cyanobacteria biomass, comprising introducing a solid phase material to the culture, result in significantly higher yields (up to 66% higher) of cyanobacteria biomass, compared to a control culture grown under the same conditions except with no solid phase material.

### Cyanobacteria

In some embodiments, the cyanobacteria are selected from one or more of Synechocystis sp. PCC 6803, Anabaena variabilis ATCC 29413, Anabaena variabilis PK84, Anabaena variabilis PK17, Crocosphaera watsonii WH 8501, Cyanothece sp. ATCC 51142, Cyanothece sp. PCC 7424, Cyanothece sp. PCC 7425, Cyanothece sp. PCC 8801, Cyanothece sp. PCC 8802, Synechococcus sp. JA-3-3Ab, Synechococcus sp. JA-2-3B′a(2-13), Microcystis aeruginosa NIES-843, Synechococcus sp. CC9311, Synechococcus sp. CC9605, Synechococcus sp. CC9902, Synechococcus sp. PCC 7002, Synechococcus sp. RCC307, Synechococcus sp. WH 7803, Synechococcus elongatus PCC 6301, Synechococcus elongatus PCC 7942, Synechococcus sp. WH 8102, Thermosynechococcus elongatus BP-1, and cyanobacterium UCYN-A. In some embodiments, the cyanobacteria is selected from Synechocystis sp. PCC 6803, or Anabaena variabilis PK84. In some embodiments, the cyanobacteria is Synechocystis sp. PCC 6803. In some embodiments, the cyanobacteria is Anabaena variabilis PK84 and other microorganisms, such as algae.

Anabaena variabilis PK84, and Anabaena variabilis PK17, are mutants of Anabaena variabilis ATCC 29413 that exhibit a high level of production of molecular hydrogen. The mutants were obtained by Shestakov et al. by chemical mutagenesis of the wild-type strain using N-methyl-N-nitro-N-nitrosoguanidine (NTG) and selection of slower growing colonies on agar medium with limited concentrations of ammonium or nitrate. PK84 with reduced activity of both hydrogenases (Hup and Hox) was previously used for cultivation in photobioreactors. See Shestakov et al., Genomic analysis of Anabaena variabilis mutants PK17 and PK84 that are characterized by high production of molecular hydrogen. Adv. Microbiol. 2013, 3, 350-365, which is incorporated herein by reference.

In some embodiments, specific species of algae serve as novel sources for the production of large quantities of long-chain insoluble hydrocarbons (alkenones) and the production of Commercial Hydrocarbons such as methane fuels and chemicals. In some embodiments, only the algae cells and not the algae oil are necessary for the production of biodiesel and green diesel, which will eliminate several costly steps for biodiesel and green diesel, such as solvent extraction.

In some embodiments, the cyanobacteria are Synechococcus elongatus PCC 7942. In one aspect, commercial kits are employed for genetic modification and expression systems for photosynthetic microalgae (Life Technologies, Algae Engineering Kits from GeneArt®).

In some embodiments, the algae are selected from one or more of any known species.

In some embodiments, the algae is alga is selected from the group consisting of green algae, red algae, eustigmatophytes, diatoms, stramenopiles, dinoflagellates, cryptomonads, euglenozoa, glaucophytes, and haptophytes.

In some embodiments, the algae are Chlamydomonas reinhardtii 137c. In one aspect, commercial kits are employed for genetic modification and expression systems for photosynthetic microalgae (Life Technologies, Algae Engineering Kits from GeneArt®).

In some embodiments, methods are provided for preparation of a pure culture of a cyanobacterial species in a medium comprising a solid phase material. In some embodiments, one or more cyanobacterial species or strains is mixed in the culture. In some embodiments, the one or more species or strains of cyanobacteria is grown in the presence of two or more species including at least one cyanobacterial species and at least one additional species selected from an algae, a non-cyanobacteria-bacterial species, or a diatom. In some embodiments, cultures of mixed species exhibit rapid growth as disclosed in U.S. Pat. No. 8,697,418 to Oyler, which is incorporated herein by reference. Oyler disclosed aquatic biomass having enhanced growth and methods for selecting and growing a mix of aquatic organisms, for example cyanobacteria and algae, to maximize production in open shallow ponds or a series of troughs. Oyler does not disclose use of a membrane or use of a cyanobacteria/E. coli culture.

### Solid Phase Material

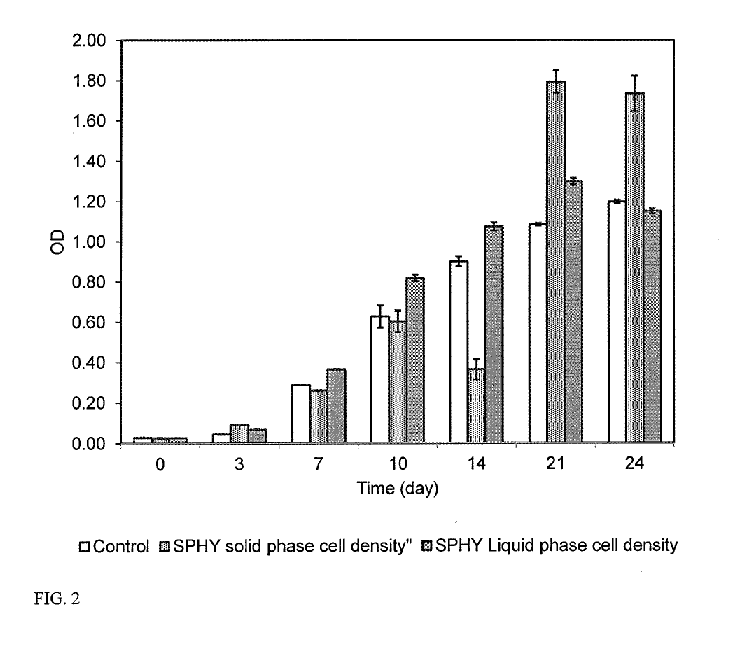

In some embodiments, a method for efficient production of cyanobacteria biomass is provided comprising growing the cyanobacteria in medium comprising a solid phase material. In some embodiments, the solid phase material is a synthetic material in the form of a porous membrane to allow for free passage of liquid medium and gasses; and with a large enough pore size to allow for passage of the cyanobacterial or algal cells. Without being bound by theory, the solid phase material surface properties, including hydrophobic properties and pore sites, result in advantageous growth of the cyanobacteria in culture. The presence of the membrane results in both higher overall yield and faster growth than the same culture without the presence of the solid phase material. The cells grow faster within the porous membrane, yet can easily be extracted by pressing the membrane. The presence of the membrane counterintuitively results in increased efficiency of the irradiated light to accelerate the growth of the cyanobacteria. For example, after three days of growth under the conditions described in example 1, using a polyethylene membrane, cells were increased by 40% within the membrane and even in the solution phase of the culture were 14% higher than under similar control conditions with no membrane. In another example, the highest cell density with solid phase material is 65% higher than the control, as shown in FIG. 2.

In some embodiments, the solid phase material is added to the culture medium before or after inoculating the culture, in one or more, two or more, three or more, or four or more, five or more, ten or more, or 20 or more, pieces of solid phase material are inserted and allowed to float freely in the culture.

In some embodiments, the solid phase material is a disposable solid phase material and is replaced for each batch, or alternatively, is reusable upon cleaning of a continuous phase system. In some embodiments, the solid phase material is a disposable solid phase material non-woven membrane that is free floating in the media and is not physically attached to the bioreactor or open pond system. In some embodiments, the membrane is a woven or non-woven membrane made of any material, so long as it is a sterilizable material. The membrane material may be sterilized by an appropriate method, depending on the material, for example, by autoclaving, irradiation, steam in place, or gaseous chemicals. The membrane may be sterilized by autoclave which subjects materials to high pressure (˜1 bar; ˜14.5 psi) saturated steam at 121 degrees C. for around 15-20 minutes. Alternatively, the membrane may be sterilized by a steam in place protocol.

In some embodiments, the solid phase material membrane is a non-woven material. In some embodiments, the solid phase material is a non-woven material selected from e.g., polyester, polyester blend, rayon, rayon blend, fiberglass, polypropylene (PP), polypropylene blend, fiberglass-polypropylene blend, fiberglass blend, polyethylene (PET), polyethylene blend; fiberglass-polyethyene blend; polypropylene-fiberglass blend; rayon-polyethylene blend; wool; wool blend; nylon; nylon blend; may be chemical/mildew and/or bacterial resistant; may be biodegradable in a landfill. In some embodiments, the non-woven solid phase is a polyester or polyester blend JR series (e.g., from Wm.T. Burnett & Co.).

In some embodiments, the solid phase material is a porous membrane with a thickness from 0.5 mm to 5,000 mm. In some embodiments, the membrane is about 1 mm to 2,000 mm thick. In some embodiments, the membrane is about 2 mm to about 1,500 mm thick. In some embodiments, the membrane is about 1,000 mm thick.

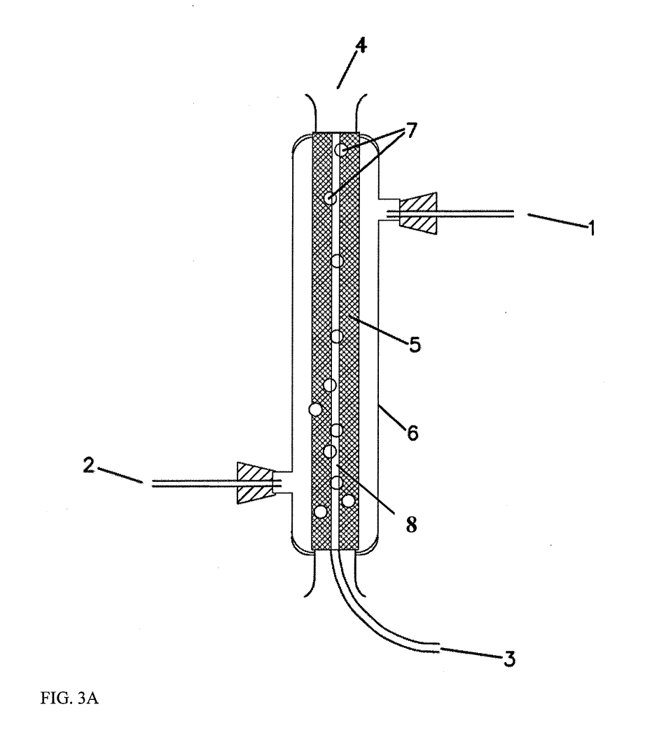

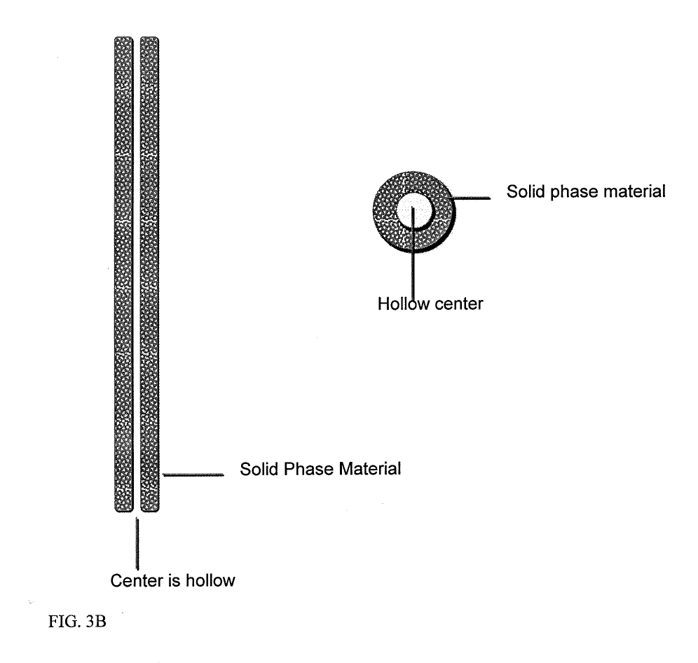

In some embodiment, the solid phase material is solidified into cylinder shape with a hollow in center (FIG. 3B). The diameter of the hollow can be lmm, 2 mm, 3 mm, 4 mm, 5 mm or larger.

In various embodiments, the solid phase material membrane may be used in a sheet, or folded or lightly compressed within the reactor to adjust for density and surface area. In some embodiments, the membrane is supplied as a cartridge. In some embodiments, the membrane is thin enough, or placed loosely enough, into the bioreactor to allow for exposure to light, substrates (minerals, nutrients and trace elements for bacteria to grow) and gas transport and exchange.

In some embodiments, the solid phase material material is a woven or knitted material. In some embodiments, the solid phase material is a woven or knitted material selected from e.g., nylon, polyester, polypropylene or PEEK porous material (e.g., SPECTRUM Labs). In some embodiments, the solid phase material material is a knotted polyester material. For example, nylon may be sterilized by irradiation; and polyester, polypropylene and PEEK are autoclavable. In some embodiments, the thickness of the woven material may be from, e.g., 45 um to 1500 um.

In some embodiments, the solid phase material is an open-cell foam material. In some embodiments, the solid phase material is an open-cell foam material selected from polyurethane, or reticulated polyurethane foam.

In some embodiments, the solid phase material is a polycellulose, or cellulose/polyester blend. In some embodiments, the solid phase material is a 55% cellulose/45% polyester nonwoven material (e.g. available from M1 Technology).

In some embodiments, the solid phase material is coated with one or more hydrophobic reagents, such as Teflon, orPolytetrafluoroethylene (PTFE), etc. In one aspect, the solid phase material is soaked in 20% Teflon solution for 5 min, then baked in 60° C. oven for 20 min. Then gradually ramp the temperature to 400° C. (e.g., 1° C./min.), and hold for 20 min, then cooling to room temperature.

### Media

In some embodiments, the method comprises use of a medium for growth of the cyanobacteria. The medium may be any medium known in the art. In some embodiments, the medium is an aqueous medium comprising water selected from the group consisting of tap water, well water, groundwater, distilled water, reverse osmosis water, sea water, rain water, grey water, river water, lake water, pond water, wastewater, and treated wastewater, and wherein the medium is filtered, unfiltered, sterilized, unsterilized, purified, or unpurified.

In some embodiments, the media may contain in whole or in part a sterilized waste stream media.

In other embodiments, the solid phase culture is grown in a medium comprising various nutrients, for example, N—P—K (Nitrogen-Phosphorus-Potassium); one or more secondary nutrients Ca (Calcium), Mg (Magnesium) and S (Sulfur); and one or more micronutrients selected from B (Boron), Cu (Copper), Fe (Iron), Cl (Chlorine), Mn (Manganese), Mo (Molybdenum), Na (Sodium), and Zn (Zinc). Basic elements such as H (Hydrogen), O (Oxygen), and C (Carbon), are derived from CO₂ (carbon dioxide) and H₂O (water), which are broken down in the process of photosynthesis to make the basic elements available for the production of carbohydrates.

In some embodiments, in particular in the case or continuous mode, the media is supplemented with an additional source of phosphorus and/or molybdenum.

In some aspects, growth can still be accelerated by adding supplemental N, thus allowing the cyanobacteria to devote more energy to biomass production and less to nitrogen fixation. Supplemental nitrogen can come from any known source. In some aspects, a nitrogen source is selected from nitrate, ammonia, urea, chitin, or chitosan, or breakdown products of chitin or chitosan, are appropriate for use as nitrogen sources with cyanobacteria or algae. See U.S. Pat. No. 8,673,619, which is incorporated by reference herein.

In some embodiments, the aqueous medium comprises one or more of water selected from the group consisting of tap water, well water, groundwater, distilled water, reverse osmosis water, sea water, rain water, grey water, river water, lake water, pond water, wastewater, and treated wastewater; nutrients N—P—K (Nitrogen-Phosphorus-Potassium); one or more secondary nutrients selected from Ca (Calcium), Mg (Magnesium) and S (Sulfur); one or more micronutrients selected from B (Boron), Cu (Copper), Fe (Iron), Cl (Chlorine), Mn (Manganese), Mo (Molybdenum), Na (Sodium), and Zn (Zinc); nitrate, ammonia, urea, chitin, or chitosan, or breakdown products of chitin or chitosan; and/or a medium selected from Aiba and Ogawa (AO) Medium, Allen Medium, Allen and Amon Medium plus Nitrate: ATCC Medium 1142, Antia's (ANT) Medium, Aquil Medium, Ashbey's Nitrogen-free Agar, ASN-III Medium, ASNIII+Turks Island Salts: CRBIP Medium 1538, ASP 2 Medium, ASW Medium: Artificial Seawater and Derivatives, ATCC Medium 617: BG-11 for Marine Blue-Green Algae; Modified ATCC Medium 616 [BG-11 medium], ATCC Medium 819: Blue-green Nitrogen fixing Medium; ATCC Medium 616 [BG-11medium] without NO3, ATCC Medium 854: ATCC Medium 616 [BG-11 medium] with Vitamin B12, ATCC Medium 1047: ATCC Medium 957 [MN marine medium] with Vitamin B12, ATCC Medium 1077: Nitrogen-fixing marine medium; ATCC Medium 957 [MN marine medium] without NO3, ATCC Medium 1234: BG-11 Uracil medium; ATCC Medium 616 [BG-11 medium] with Uracil, Beggiatoa Medium: ATCC Medium 138, Beggiatoa Medium 2: ATCC Medium 1193, Blue-Green (BG) Medium, BG-11 Medium for Blue Green Algae: ATCC Medium 616, BG11+ASNIII (10%): CRBIP Medium 1540, BG11+ASNIII (1:1): CRBIP Medium 1546, BG11+NaHCO3: CRBIP Medium 1547; BGI I+Turks Island Salts (25%)+NaHCO3:CRBIP Medium 1548, Bold's Basal (BB) Medium, Bold 1NV Medium, Bold 3N Medium, Bristol Medium, Castenholtz D Medium, Castenholtz D Medium Modified: Halophilic Cyanobacteria, Castenholtz DG Medium, Castenholtz DGN Medium, Castenholtz ND Medium, Chloroflexus Broth, Chloroflexus Medium: ATCC Medium 920, Chu's #10 Medium: ATCC Medium 341, Chu's #10 Medium Modified, Chu's #11 Medium Modified, COMBO Medium Modified, CR1 Soil, Cyanophacyean Medium, DCM Medium, DYIV Medium, E27 Medium, E31 Medium and Derivatives, Erd-Schreiber 2× Medium, f/2 Medium, f/2 Medium Derivatives, Fraquil Medium: Freshwater Trace Metal-Buffered Medium, Gorham's Medium for Algae: ATCC Medium 625, h/2 Medium, Jansen's (J) Medium, Jaworski's (JM) Medium, K Medium, L1 Medium and Derivatives, MN Marine Medium: ATCC Medium 957, Plymouth Erdschreiber (PE) Medium, Prochlorococcus PC Medium, Prochlorococcus Medium: CRBIP Medium 1559, Pro99 Medium, Proteose Peptone (PP) Medium, Prov Medium, Prov Medium Derivatives, S77 plus Vitamins Medium, S88 plus Vitamins Medium, Saltwater Nutrient Agar (SNA) Medium and Derivatives, SES Medium, SN Medium, Modified SN Medium, SNAX Medium, Soil/Water Biphasic (S/W) Medium and Derivatives, SOT Medium for Spirulina: ATCC Medium 1679, Spirulina (SP) Medium, van Rijn and Cohen (RC) Medium, Walsby's Medium, YBC-II Medium, Yopp Medium, and Z8 Medium. Recipes for the media can be found at See http://www-cyanosite.bio.purdue.edu/media/table/media.html, which is incorporated herein by reference.

In some embodiments, the medium is BG-11 medium ATCC 616.

### Bioreactors

In some embodiments, any bioreactor means known in the art can be employed in the methods provided herein. In some embodiments, the bioreactor is a photobioreactor. In some embodiments, the bioreactor is a photobioreactor fitted with means for introducing CO₂, light and media. In some embodiments, the photobioreactor has a means for agitation of the culture medium.

In some embodiments, the photobioreactor is an air lift tubular design or flat tank. In some embodiments, a commercial-scale photobioreactor is employed (20 L to >100,000 L). In some embodiments, the photobioreactor comprises transparent surfaces, and is configured to allow high mass transfer rates and production of large biomass yields.

In some embodiments, the photobioreactor is a commercial model, for example, selected from a 2-20 liter autoclavable stirred tank photo bioreactor, or a rocking, single use 10-50 L bioreactor (e.g. Applikon Biotechnology) fitted with, e.g. solid state light emitting diodes (LEDs).

In some embodiments, the inoculated culture medium in a bioreactor is illuminated for part of or a continuous 24 hour day with PAR wavelengths, for example, by LED lighting (internally or externally) can be employed.

In some embodiments, the cyanobacteria or algae growth is monitored by an attached process control device to measure OD 710 nm which is correlated with cell numbers by prior calculation. In some embodiments, the photobioreactor comprises a device to drain or harvest the cells on a regular basis, for example, a daily basis.

In some embodiments, the photobioreactor comprises LED light sources inserted into the center, to provide a 360 degree angle illumination to maximise the growth of different cyanobacteria or algae strains.

In some embodiments, the method further comprises determination of peak exponential growth from monitoring the photobioreactor.

For example, Sathiyamoorthy et al. developed a low cost inexpensive system made of a polypropylene bag which could hold 20-40 liters of media and be exposed to a light source. The yield was reported to be comparable to that from large, open-air systems. The advantage was the ability to prepare contamination-free cultures at affordable cost. See Sathiyamoorthy et al., 1994, A low-cost bioreactor for cyanobacterial biomass production, Bioresource Technology, 49, 3, 279-280, which is incorporated herein by reference.

In some embodiments, the bioreactor is a flat panel bioreactor system, for example, as shown in Wijffels et al, 2010 An outlook on microalgael fuels, 13 Aug. 2010, vol. 329, Science pp. 796-799, which is incorporated herein by reference.

In some embodiments, the bioreactor is fitted with a light source such as a photodiode array. For example, Melnicki et al. 2013 developed a 7.5 L, aluminum-clad feedback controlled LED photobioreactor housing quantum sensors and light emitting diode arrays, which provided 630 or 680 nm light to preferentially excite the major cyanobacterial pigments, phycocyanin or chlorophyll a, respectively. The bioreactor was also fitted with a turbidostat to adjust media dilution to optimize growth. See Melnicki et al., Feed-back controlled LED photobioreactor for photophysiological studies of cyanobacteria, Bioresource Technology, April 2013, 127-133, which is incorporated herein by reference.

In one embodiment, on reaching peak exponential growth 50% of the cells are harvested daily to a separate batch holding tank which has aeration (ambient air mix) attached and LED lighting 24/7. The algal strains are held under prescribed conditions for 3-4 days and samples from the tank monitored visually under a microscope daily for exopolysaccharide production. These cells are finally extracted for example, by dewatering for either carbohydrate extraction or for the uses described earlier.

### Open Pond Systems

In some embodiments, methods are provided for growing lipid-forming algae or cyanobacteria in open ponds in the presence of a solid phase material as described herein. Outdoor open pond growth systems can be illuminated by natural sunlight. In some embodiments, the method comprises use of an open pond system for mass production of cyanobacteria or algae. A report from the National Renewable Energy Laboratory 1998, stated that open pond systems 1,000 m² pond systems built in California, Hawaii, and New Mexico could be run with high efficiency CO₂ utilization with careful control of pH and other physical conditions allowed 90% utilization of injected CO₂. Production levels were stated to reach up to 50 g per square meter per day, but consistency was hampered by low temperature conditions, especially at night. See Sheehan et al., 1998. It is anticipated that use of solid phase material will result in enhanced production levels in an open pond system to exceed 50 g per m² per day. In some embodiments, an open pond system is employed with means for CO₂ injection, pH control and optionally temperature control.

In some embodiments, the solid phase material is collected from the bioreactor, or open pond system, and the biomass is harvested from the solid phase material, for example, by expressing the cyanobacteria or algae from the solid phase material.

### Light Source

In some embodiments, the innoculated medium is illuminated with light selected from the group consisting of sun light, artificial light, and mixtures thereof.

In some embodiments, the bioreactor is fitted with or exposed to a light source. In various embodiments, the light source is ambient light and/or irradiated light. In some embodiments, the light source is selected from natural sunlight, fluorescent light, incandescent light, or LED. In some embodiments, the light source is a consistent fluorescent light intensity. In some embodiments, the light source is cycled to mimic daylight cycles. In some embodiments, the irradiated light includes a wavelength or range between about 350 nm to 750 nm in wavelength. In some embodiments, the irradiated light includes a wavelength or range of Photosynthetically Active Radiation, PAR, between about 400 nm to 700 nm wavelength. In some embodiments, the light source is capable of emitting light in the wavelengths of about 450-500 nm and 630-700 nm. In some embodiments, the light source is capable of emitting light in every PAR spectral range and having peaks at 470 nm and 630 nm to mimic natural sunlight.

In some embodiments, the method further comprises monitoring and/or adjusting the pH to allow for optimal conditions for growth of biomass. Optimal pH conditions are dependent upon the selection of the cyanobacteria or algae selected for use in the methods provided herein. In some embodiments, the pH is adjusted with addition of bicarbonate, carbonate, NaOH, and/or HCl. In some embodiments, the pH in the culture medium is adjusted to between about pH 5 to about pH 10, pH 6 to about pH 9, or pH 5 to about pH 8, or about pH 7±1. In other embodiments, high alkalinity is optimal, for example, for the growth of Spirulina plantensis. In some embodiments, the pH is maintained between about pH 6 to about pH 12; or about pH 7 to about pH 11, or about pH 8 to about pH 9, or about pH 9±1. In some embodiments, the photobioreactor, or open pond system, has a means for monitoring and adjusting pH.

### Temperature

In some embodiments, the temperature of the bioreactor is selected from a temperature within about 5 degrees C. to about 40 degrees C., about 10 degrees C. to about 37 degrees C., or about 15 degrees C. to about 35 degrees C. In some embodiments, the method is performed wherein the media temperature is about 20 to about 30 degrees C. In some embodiments, the media temperature is about 25 degrees C. In some embodiments, the media temperature is about 32±5 degrees C. In some embodiments, the media temperature is about 30±5 degrees C. In some embodiments, the media temperature is about 30±3 degrees C. In some embodiments, the media temperature is about 30±1 degrees C. In some embodiments, the temperature is ambient temperature.

### Carbon Dioxide

In some embodiments, the methods require an external supplementation of carbon dioxide (CO₂). In various embodiments, the carbon dioxide is from a source selected from the group consisting of air, enriched carbon dioxide, pure carbon dioxide, flue gas, combustion gas, and fermentation gas. In some aspects, the source of CO₂ can be combustion waste, industrial off line gas or greenhouse gases etc. In some embodiments, CO₂ is bubbled through the culture media. In some embodiments, the CO₂ is bubbled through the solid phase material in the culture media. In other embodiments, no additional CO₂ is added to the media.

### Processing

In some embodiments, a method for efficient production of cyanobacteria or algal biomass is provided comprising growing the cyanobacteria or alga in a bioreactor in media comprising a solid phase material membrane.

In some embodiments, the cyanobacteria are harvested from the bioreactor and processed by any means known in the art. For example, in some aspects, fatty acids from lipid-rich cyanobacteria biomass are converted chemically to other products such as biodiesel. Generally, alkanes C₅₋₉ can be used as gasoline components, while alkanes C₈₋₂₁ can be used as diesel components.

In some embodiments, on the laboratory scale experiments, cells are extracted from the solid phase material by pressing. On the bioreactor scale, cells can be harvested from the membrane by any means, such as physical a plunger, roller, paddle, etc., either incorporated to the bioreactor, or alternatively after removing the membrane from the bioreactor, for example, through the effluent port; then extracting the membrane. The process of solid phase extraction has higher efficiency and more environmental friendly than traditional liquid phase concentrating/dewatering or chemical extraction.

In some embodiments, genetically manipulated cyanobacteria are produced using the solid phase based techniques provided herein to produce secretable products directly, for example, ethanol, ethylene, isoprene, free fatty acids, fatty alcohols, isobutyraldehyde, 1-butanol, or hydrogen.

FIG. 3A shows a schematic of a photobioreactor for solid phase based production of cyanobacteria. The bioreactor is fitted with a CO₂ gas influent port 3 and CO₂ effluent port 1. A media inlet port 2 and media and CO₂ effluent port 1 are provided. In the present embodiment, a solid phase material 5 is immersed in the media within the core of the bioreactor and the solid phase material runs the length of the core of the bioreactor. In some embodiments, the shell 6 of the bioreactor is made of a transparent or transluscent material to allow for an external light source. In other embodiments, the shell 6 of the bioreactor is opaque, such as a metallic shell of stainless steel or aluminum, and the core of the bioreactor 8 is fitted with a light source. In some embodiments, H₂ gas 7 is bubbled through the bioreactor media and collected through port 4. In various embodiments, the bioreactor can be run in either batch mode, semi-continuous mode or continuous mode. FIG. 3B shows two views of a cylindrical geometric design of the bioreactor solid phase material with a hollow center.

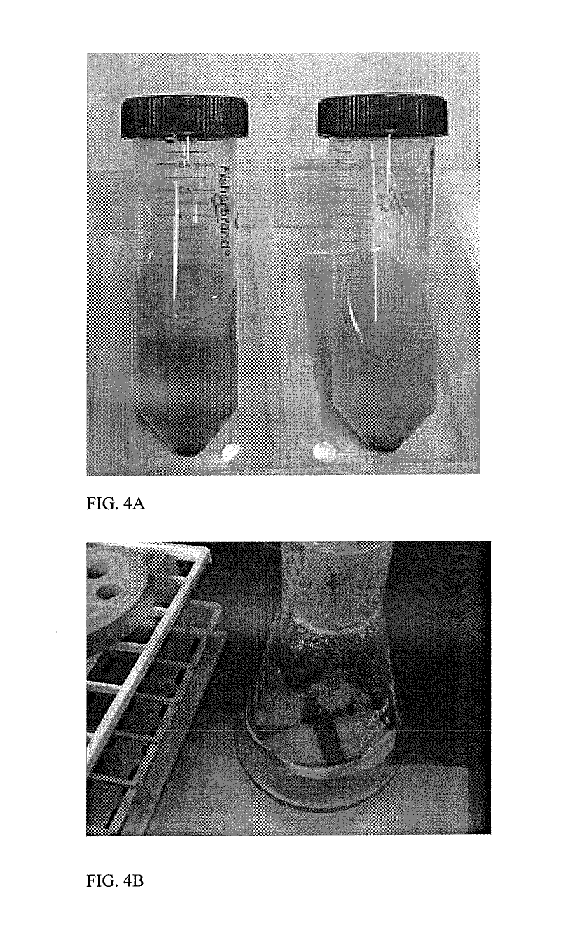

FIG. 4A shows a photograph of pure Cyanobacteria Synechocystis PCC 6803 culture prepared according to Example 1. Cells grow in higher yield using the solid phase based high yield technique according to example 1 on the left employing a sterile polyethylene 2 mm thick×2 cm×2 cm solid phase material; compared to control culture under identical conditions, but without the solid phase material, on the right. Both tubes were treated under the same conditions for three days, measuring optical density periodically. After 3 days, in the solid phase portion of the culture, cells have 40% higher yield than the control. After 3 days, in the liquid phase portion of the culture, cells have 14% higher yield than the control.

FIG. 4B shows an example of one mode, wherein the culture is grown in a 250 mL Erlenmeyer flask with four pieces of solid phase material are inserted and allowed to float freely in the culture. The flask is shown partially submerged in an incubator water bath with exposure to light from above.

### Examples

Medium

BG-11 medium ATCC 616 was prepared as follows and sterilized. The medium was adjusted to pH 7.1 after sterilization.

| Material | Amount | |
|---|---|---|
| NaNO₃ | 1.5 | g |
| K₂HPO₄ | 0.04 | g |
| MgSO₄•7H₂O | 0.075 | g |
| Citric acid | 0.006 | g |
| Ferric ammonium citrate | 0.006 | g |
| EDTA (disodium salt) | 0.001 | g |
| NaCO₃ | 0.02 | g |
| Trace metal mix A5 | 1.0 | ml |
| Agar (if needed) | 10.0 | g |
| Distilled water | 1.0 | L |

### Trace Metal Mix A5:

| Material | Amount | |
|---|---|---|
| H₃BO₃ | 2.86 | g |
| MnC1₂•4H₂O | 1.81 | g |
| ZnSO₄•7H₂O | 0.222 | g |
| NaMoO₄•2H₂O | 0.39 | g |
| CuSO₄•5H₂O | 0.079 | g |
| Co(NO₃)₂•6H₂O | 49.4 | mg |
| Distilled water | 1.0 | L |

### Example 1

### Solid Phase Pure Cyanobacteria Synechocystis PCC 6803 Culture Preparation

Cyanobacteria Synechocystis sp. strain PCC 6803 was obtained from ATCC.

Protocol.

Dilute media BG-11 to 1× in filtered DI water. Transport 30 ml 1× media in sterilized corning tube.

Inoculate 10% (v/v) Cyanobacteria Synechocystis sp. strain PCC 6803 in media, and vortex the culture for 10 s.

Insert 2 cm×2 cm×1 cm sterilized membrane into the culture and shake until uniform.

Wrap the top of the tube with hydrophobic filter paper or parafilm with evenly punched pores.

Insert tubes in 30° C. incubator shaker.

Fluorescent light intensity was employed.

Experiment.

Sterilized 2 of hollow membrane 2×2 cm and 2 of foam membrane 2×2 cm.

Added one membrane each into autoclaved tubes into which 20 ml GB-11+5 ml inoculate from 07/07 and 07/13 cultures had been added as follows:

20 ml BG-11+inoculate with foam membrane

20 ml BG-11+inoculate from 20 ml BG-11+inoculate polyethylene membrane

20 ml BG-11+inoculate=control blank (no membrane).

Samples and control were propagated under constant temperature (30° C.) in incubator and optical density (OD 710 nm) was measured periodically (3 days) for analysis over a period of 9 days then stored at 4° C. afterwards. After three days representative tubes of polyethylene membrane and control are shown in FIG. 4A. Both tubes were treated under the same conditions for nine days, measuring optical density periodically. After 3 days, in the solid phase portion of the culture, cells have 40% higher yield than the control. After 3 days, in the liquid phase portion of the culture, cells have 14% higher yield than the control. Visual observation revealed the solid phase culture appeared dark green with more suspended cells and dark green precipitate. The control culture exhibited dark green precipitate, but fewer suspended cells as evident from lighter colored solution.

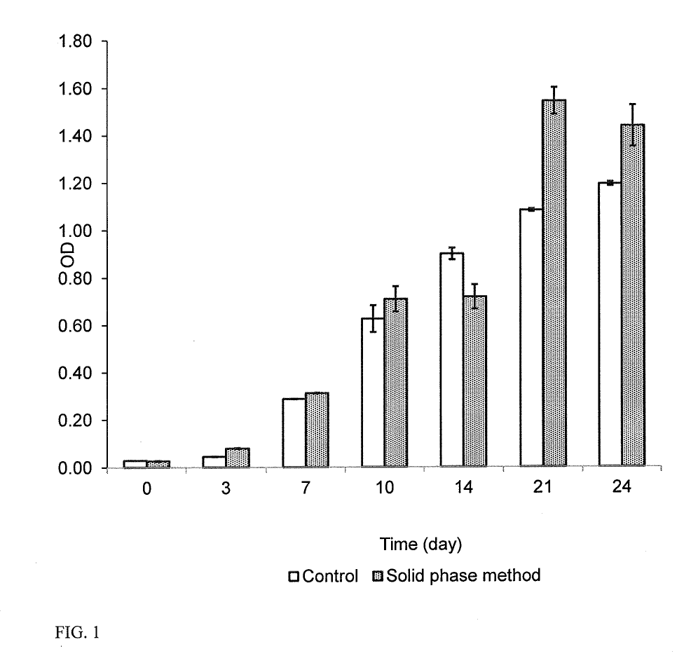

FIG. 1 shows a comparison of time-lapse cell density of Solid Phase based High Yield method (SPHY) to control. The control refers to the cultures that are without solid phase. The results of SPHY combine the cell density in solid phase and in liquid phase of the culture. SPHY dimension is 20 mm×20 mm×5 mm. At day 18, solid phase material was taken out of the culture vessel and treated by purging with CO₂ for 1 hour while the control stays the same condition. At day 21, the solid phase material has 65% higher yield than control. If combine the enhancement of the solid and liquid phase, the yield is 42% higher than control. All samples are propagated under the same conditions. Blank was deducted from all data. Error bars were calculated at the 95% confidence interval.

### Example 2

### Mixed Culture Preparation

In some embodiments, the cyanobacterial culture is a mixed culture comprising cyanobacteria and an additional microbial species. In any case, a solid phase material is employed as described above. The following protocol describes a mixed culture of Cyanobacteria Synechocystis sp. strain PCC 6803 and an E. coli secondary inoculum.

Protocol.

Dilute BG-11 media to 1× in filtered DI water. Transport 30 ml 1× media in sterilized corning tube.

Inoculate 10% (v/v) Cyanobacteria Synechocystis sp. strain PCC 6803 in media, and vortex the culture for 10 s.

Insert 2 cm×2 cm×1 cm sterilized membrane into the culture, then shake uniform.

Inject equal volume of E-coli (Secondary inoculum) inside of the membrane.

Wrap the top of the tube with hydrophobic filter paper or parafilm with evenly punched pores.

Insert tubes in 30° C. incubator shaker.

### Example 3

### Bioreactor Modes

The culture preparation can be scaled up into a bioreactor with the solid phase material and run in a variety of modes as follows and run in either batch or flow through modes with or without CO₂.

1, Media batch and no CO₂ flow through.

2, Media batch with CO₂ flow through.

3, Media flow though with no CO₂ flow though.

4, Media flow through with CO₂ flow through.

### Example 4A

### Growth Agar Preparation

Cyanobacteria stains of interest are cultivated in the laboratory with periodic passages on agar slices and plates.

1 L Agar for Freshwater Cyanobacteria (No Glucose)

This protocol produces 1 L agar suitable for plating freshwater cyanobacteria (PCC7942 and PCC6803).
Mix 10 g agar and 1 mM thiosulfate (=0.248 g), top off with H₂O to 500 mL total volume.

1. Autoclave the product of (1).

2. Mix 20 mL 50× BG-11 solution and 480 mL of H₂O.

3. Autoclave the product of (3).

4. Mix (2) and (4), pour plates and let cool.

1 L Agar for Freshwater Cyanobacteria (with Glucose)

This protocol produces 1 L agar with 5 mM glucose suitable for plating PCC6803 cyanobacteria.
1. Mix 10 g agar and 1 mM thiosulfate (=0.248 g), top off with H₂O to 500 mL total volume.
2. Autoclave the product of (1).
3. Mix 20 mL 50× BG-11 solution and 230 mL of H₂O.
4. Autoclave the product of (3).
5. Mix 5 mL glucose and 245 mL of H₂O.
6. Autoclave the product of (5).
7. Mix (2) and (4) and (6), pour plates and let cool.

### Example 4B

### Frozen Stock Solutions

Two liquid cultures of the syn pcc 6803 strain were started on different dates 07/07 and 07/13 and grown as follows. Placed 0.2 μm filtered DI water, 19.6 ml+0.4 ml (50×) GB11 into 4 autoclaved 50 mL centrifuge tubes. Added syn pcc 6803 and put in 30° C. incubator shake at 1. Inoculated 3 plates with syn. Again put 30° C. incubator shake at 1. Six days after starting the second liquid culture, cells growth was apparent and culture appeared dark green in both 07/07 and 07/13 samples. Optical density (OD) of cultures was measured. Visual appearance was described as follows.
OD 07/07 darker uniform suspension
   07/13 dark green coagulated suspension

Frozen stocks are made by centrifugation of 2 tubes of cyanobacteria culture after several days of growth. Cells are resuspended in 4 ml fresh BG-11. Aliquot 0.5 ml of resuspended stock into 8 autoclaved tubes containing 40 □L of DMSO and frozen instantly. The vial is thawed by rolling in hands. Strike a petri dish from the frozen stock upon thawing. Inocula is taken from thawed frozen stock as needed and put in 15 ml BG-11, incubate at 30° C. and move to a shaker after 1-3 days; observe growth periodically (triplicates).

### Example 5

### Solid Phase Pure Cyanobacteria Synechocystis PCC 6803 Culture Preparation with CO₂ Dissolved in the Solid Phase

Samples and control were propagated under the conditions shown in Example 1.

Sample tubes: 20 ml BG-11+inoculate with polyethylene membrane.

Control tubes: 20 ml BG-11+inoculate=control blank (no membrane).

The sample and control tubes were grown at constant temperature (30° C.) and light conditions with non-woven polyethylene SPHY dimension of 20 mm×20 mm×5 mm. The same conditions, culture medium and inocula were employed as shown in Example 1. Cultures were grown in an incubator for a period of 24 days. Results are shown in FIGS. 4 and 5. At day 18, solid phase material was taken out of the culture vessel and the solid phase material was treated by purging with CO₂ for 1 hour while the control stayed the same condition. The solid phase was returned to the vessel afterwards. All samples were otherwise propagated under the same conditions. Optical density was measured periodically.

In detail, the CO₂ is generated through thermal decomposition of sodium bicarbonate heated in sterile deionized water.

2 NaHCO₃(s)→CO₂(g)+H₂O(g)+Na₂CO₃(s)

The CO₂ treatment is performed in a 250 ml flat bottom triangular Erlemmeyer flask, as shown in FIG. 4B, containing 100 ml of DD H₂O sterilized by filtering through a 0.1 micron filter. The solid phase membranes are transferred from the culture tubes into the sterile DD H2O in the triangular flask using a sterile pincer. The triangular flask is placed in a water bath with the temperature maintained at 70° C. (switch set at 6.4). 10 g of sodium bicarbonate powder is added into the sterile DD H₂O in the beginning. After the bubbles start to generate, 5 g of sodium bicarbonate powder is added every 20 min for 1 hr to generate CO₂ bubbles in a consecutive manner. During the treatment, the solid phase membranes are surrounded by the CO₂ bubbles.

FIG. 1 shows a comparison of time-lapse cell density of overall Solid Phase based High Yield method (SPHY) to control. The control (open bars) refers to the cultures that are without solid phase. The results of SPHY (shaded bars) combined the cell density in solid phase and in liquid phase of the culture. Blank was deducted from all data. Error bars were calculated at the 95% confidence interval. The solid phase sample cultures exhibited consistently higher OD than control over the first ten days of growth. Growth in the solid phase lagged at day 14. Following only 1 h of CO₂ exposure at day 18, the overall solid phase culture exhibited significantly greater cell density than the control culture at days 21 and 24.

FIG. 2 shows a comparison of time-lapse cell density of solid and liquid phases of the Solid Phase based High Yield method (SPHY) to control. The control (open bars) refer to the cultures that are without solid phase. The results of cell density of SPHY in solid phase (shaded bars) were separated from liquid phase (solid bars). All samples are propagated under the same conditions. Blank was deducted from all data. Error bars were calculated at the 95% confidence interval. Following only 1 h of CO₂ exposure at day 18, the solid phase portion of the SPHY culture exhibited significantly greater cell density than the control culture at days 21 and 24.

### REFERENCES

Wang, Weihua; Liu, Xufeng; Lu, Xuefeng, Engineering cyanobacteria to improve photosynthetic production of alka(e)nes, Biotechnology for Biofuels 2013, 6:69 published 6 May 2013.
Sergey V. Shestakov, Lidia E. Mikheeva, Andrey V. Mardanov, Nikolai V. Ravin, Konstantin G. Skryabin, Genomic analysis of Anabaena variabilis mutants PK17 and PK84 that are characterized by high production of molecular hydrogen. Adv. Microbiol. 2013, 3, 350-365.
Liu, Jie, Lei Chen, Jiangxin Wang, Jianjun Qiao and Weiwen Zhang, Proteomic analysis reveals resistance mechanism against biofuel hexane in Synechocystis sp. PCC 6803, Biotechnology for Biofuels 2012, 5:68
Sathiyamoorthy et al., A low-cost bioreactor for cyanobacterial biomass production, Bioresource Technology, 1994, 49, 3, 279-280.
Kim, Hyun Woo et al., 2010, Photoautotrophic nutrient utilization and limitation during semi-continuous growth of Synechocystis sp. PCC6803, Biotechnology and Bioengineering, 106, 4, 553-563 July 2010.
Sheehan, J., Dunahay, T., Benemann, J. and Roessler, P. G. (1998) US Department of Energy's Office of Fuels Development, July 1998. A Look Back at the US Department of Energy's Aquatic Species Program—Biodiesel from Algae, Close Out Report TP-580-24190. Golden, Colo.: National Renewable Energy Laboratory.
Pandey, J. P. et al., 2010, Standardization of pH and light intensity for the biomass production of Spirulina platensis, J Algal Biomass Utln, 2010, 1(2):93-102.
Markou, Giorgos; Georgakakis, Dimitris, 2011, Cultivation of filamentous cyanobacteria (blue-green algae) in agro-industrial wastes and wastewaters: a review, Applied Energy 88(2011) 3389-3401.
U.S. Pat. No. 8,697,418, issued Apr. 15, 2014; Use of mixed species for rapid growth of aquatic biomass, Oyler; James R.
U.S. Pat. No. 8,728,783, issued May 20, 2014; Photobioreactor, Aikens, John; Turner, Robert J.

We claim:

1. A method for increasing cell density associated with faster rate of cell growth and/or replication of microorganisms selected from cyanobacteria or algae biomass in an aqueous culture medium, the method comprising
introducing a solid phase material into the aqueous culture medium; and
inoculating the culture medium with cyanobacteria or algae.

2. The method according to claim 1, wherein the solid phase material is introduced after inoculating the culture medium with the cyanobacteria or algae.

3. The method according to claim 1, wherein the solid phase material is introduced to the culture medium prior to inoculating the culture medium with the cyanobacteria or algae.

4. The method according to claim 1, wherein the inoculating comprises introducing the cyanobacteria or algae into the aqueous culture medium.

5. The method according to claim 1, wherein the inoculating comprises introducing the cyanobacteria or algae into the solid phase material infused with culture medium.

6. The method according to claim 1 further comprising exposing the inoculated culture medium to light to one or more wavelength ranges in the PAR spectral range comprising light between 400 nm to 770 nm, 400 nm to 700 nm, 450 nm to 500 nm, 630 nm to 700 nm, every PAR spectral range having peaks at 470 nm and 630 nm, 500 nm to 665 nm having peaks around 660 nm, and/or 440 nm to 500 nm, preferably around 460 nm.

7. The method according to claim 1 wherein the cyanobacteria is selected from one or more of the group consisting of Synechocystis sp. PCC 6803, Anabaena variabilis ATCC 29413, Anabaena variabilis PK84, Anabaena variabilis PK17, Crocosphaera watsonii WH 8501, Cyanothece sp. ATCC 51142, Cyanothece sp. PCC 7424, Cyanothece sp. PCC 7425, Cyanothece sp. PCC 8801, Cyanothece sp. PCC 8802, Synechococcus sp. JA-3-3Ab, Synechococcus sp. JA-2-3B′a(2-13), Microcystis aeruginosa NIES-843, Synechococcus sp. CC9311, Synechococcus sp. CC9605, Synechococcus sp. CC9902, Synechococcus sp. PCC 7002, Synechococcus sp. RCC307, Synechococcus sp. WH 7803, Synechococcus elongatus PCC 6301, Synechococcus elongatus PCC 7942, Synechococcus sp. WH 8102, Thermosynechococcus elongatus BP-1, Spirulina spp., and cyanobacterium UCYN-A.

8. The method according to claim 7 wherein the cyanobacteria is selected from one or more of the group consisting of Synechocystis sp. PCC 6803, Anabaena variabilis ATCC 29413, Anabaena variabilis PK84, and Anabaena variabilis PK17.

9. The method according to claim 1, wherein the algae are selected from the group consisting of green algae, red algae, eustigmatophytes, diatoms, stramenopiles, dinoflagellates, cryptomonads, euglenozoa, glaucophytes, and haptophytes.

10. The method according to claim 1, wherein the cyanobacteria or algae is present in a mixed culture with one or more of other bacteria, selected from E. coli, shewanella, or alteromonas.

11. The method according to claim 1, wherein the membrane is selected from a porous non-woven, woven, knitted, or foam material.

12. The method according to claim 11, wherein the porous non-woven material is selected from polyethylene, polyethylene blend, polyester, polyester blend, rayon, rayon blend, fiberglass, polypropylene (PP), polypropylene blend, fiberglass-polypropylene blend, fiberglass blend, polyethylene (PET), polyethylene blend; fiberglass-polyethyene blend; polypropylene-fiberglass blend; rayon-polyethylene blend; wool; wool blend; nylon; or nylon blend.

13. The method according to claim 11, wherein the woven or knitted material is elected from nylon, polyester, polypropylene or PEEK porous material.

14. The method according to claim 11, wherein the foam material is open-cell foam material selected from polyurethane, or reticulated polyurethane foam.

15. The method according to claim 1 wherein the medium comprises one or more of wastewater, Aiba and Ogawa (AO) Medium, Allen Medium, Allen and Arnon Medium plus Nitrate: ATCC Medium 1142, Antia's (ANT) Medium, Aquil Medium, Ashbey's Nitrogen-free Agar, ASN-III Medium, ASNIII+Turks Island Salts: CRBIP Medium 1538, ASP 2 Medium, ASW Medium: Artificial Seawater and Derivatives, ATCC Medium 617: BG-11 for Marine Blue-Green Algae; Modified ATCC Medium 616 [BG-11 medium], ATCC Medium 819: Blue-green Nitrogen fixing Medium; ATCC Medium 616 [BG-11medium] without NO3, ATCC Medium 854: ATCC Medium 616 [BG-11 medium] with Vitamin B12, ATCC Medium 1047: ATCC Medium 957 [MN marine medium] with Vitamin B12, ATCC Medium 1077: Nitrogen-fixing marine medium; ATCC Medium 957 [MN marine medium] without NO3, ATCC Medium 1234: BG-11 Uracil medium; ATCC Medium 616 [BG-11 medium] with Uracil, Beggiatoa Medium: ATCC Medium 138, Beggiatoa Medium 2: ATCC Medium 1193, Blue-Green (BG) Medium, BG-11 Medium for Blue Green Algae: ATCC Medium 616, BG11+ASNIII (10%): CRBIP Medium 1540, BG11+ASNIII (1:1): CRBIP Medium 1546, BG11+NaHCO3: CRBIP Medium 1547; BG11+Turks Island Salts (25%)+NaHCO3:CRBIP Medium 1548, Bold's Basal (BB) Medium, Bold 1NV Medium, Bold 3N Medium, Bristol Medium, Castenholtz D Medium, Castenholtz D Medium Modified: Halophilic Cyanobacteria, Castenholtz DG Medium, Castenholtz DGN Medium, Castenholtz ND Medium, Chloroflexus Broth, Chloroflexus Medium: ATCC Medium 920, Chu's #10 Medium: ATCC Medium 341, Chu's #10 Medium Modified, Chu's #11 Medium Modified, COMBO Medium Modified, CR1 Soil, Cyanophacyean Medium, DCM Medium, DYIV Medium, E27 Medium, E31 Medium and Derivatives, Erd-Schreiber 2× Medium, f/2 Medium, f/2 Medium Derivatives, Fraquil Medium: Freshwater Trace Metal-Buffered Medium, Gorham's Medium for Algae: ATCC Medium 625, h/2 Medium, Jansen's (J) Medium, Jaworski's (JM) Medium, K Medium, L1 Medium and Derivatives, MN Marine Medium: ATCC Medium 957, Plymouth Erdschreiber (PE) Medium, Prochlorococcus PC Medium, Prochlorococcus Medium: CRBIP Medium 1559, Pro99 Medium, Proteose Peptone (PP) Medium, Prov Medium, Prov Medium Derivatives, S77 plus Vitamins Medium, S88 plus Vitamins Medium, Saltwater Nutrient Agar (SNA) Medium and Derivatives, SES Medium, SN Medium, Modified SN Medium, SNAX Medium, Soil/Water Biphasic (S/W) Medium and Derivatives, SOT Medium for Spirulina: ATCC Medium 1679, Spirulina (SP) Medium, van Rijn and Cohen (RC) Medium, Walsby's Medium, YBC-II Medium, Yopp Medium, and Z8 Medium, and M9 medium.

16. The method according to claim 15, wherein the medium is BG-11 medium ATCC 616.

17. A method for increasing growth rate and/or yield of cyanobacteria biomass in an aqueous culture medium, the method comprising
introducing a solid phase material into the aqueous culture in a photobioreactor or an open pond system.

18. The method according to claim 17, wherein the photobioreactor is fitted with a means for providing irradiated light.

19. The method according to claim 17, wherein the photobioreactor is fitted without providing irradiated light.

20. The method according to claim 17, wherein the photobioreactor is fitted with a means for providing irradiated light including between about 400 nm to 770 nm wavelength.

21. The method according to claim 17, wherein the photoreactor is fitted with a means for introducing carbon dioxide periodically or continuously to the aqueous culture.

22. The method according to claim 17, wherein the aqueous medium comprises water selected from the group consisting of tap water, well water, groundwater, distilled water, reverse osmosis water, sea water, rain water, grey water, river water, lake water, pond water, and wastewater.
